(19) United States
(12) Patent Application Publication
(10) Pub. No.: US 2014/0302108 A1
(43) Pub. Date: Oct. 9, 2014

(54) Nail Discoloration and Fungus Treatment

(71) Applicant: Spenco Medical Corporation, Waco, TX (US)

(72) Inventor: Karen Carmody Smith, Lorena, TX (US)

(73) Assignee: Spenco Medical Corporation, Waco, TX (US)

(21) Appl. No.: 14/287,402

(22) Filed: May 27, 2014

Related U.S. Application Data
(62) Division of application No. 13/695,424, filed on Oct. 30, 2012.
(60) Provisional application No. 61/330,680, filed on May 3, 2010.

(51) Int. Cl. A61K 33/38

(52) U.S. Cl. CPC A61K 33/38
USPC 424/405; 424/618

(57) ABSTRACT

A system and method for treating human nails afflicted with fungus is now disclosed. The system comprises a hydrogel dressing or pad, a topical agent consisting of a nanosilver treatment agent, a topical carrier in which said treatment agent is dispersed, and an adhesive for securing the hydrogel and treatment agent to a nail in need of such treatment. A kit, method and regimen for treatment of the nail are also disclosed.

### CROSS REFERENCE TO RELATED APPLICATIONS

This application is a divisional of U.S. patent application Ser. No. 13/695,424, filed on Oct. 30, 2012, which is the U.S. National Stage Application of International Application No. PCT/US2011/035046, filed on May 3, 2011, which claims the benefit of U.S. Provisional Patent Application Ser. No. 61/330,680 filed on May 3, 2010.

### STATEMENT REGARDING FEDERALLY SPONSORED RESEARCH OR DEVELOPMENT

None.

### TECHNICAL FIELD OF INVENTION

This invention relates to the field of systems and methods for treating humans having toenails or fingernails afflicted with fungus and/or which are discolored or unusually thickened.

### BACKGROUND OF THE INVENTION

Nail fungus (also called onychomycosis of the nail plate or tinea of the nails) is a difficult condition to eradicate. In this condition, which may be of concern primarily to human patients, fungus grows under the nail plate which ironically provides a protective covering for the fungus and makes it difficult for medicaments to attack the fungus. The condition may affect both toenails and fingernails, although it is more prevalent in toenails since the wearing of shoes provides a dark, moist environment favored by fungal organisms.

Various systematic and topical treatment agents have been reported for the treatment of nail fungus. Systematic agents, pharmaceutical compositions taken orally by a patient, may be contraindicated for certain patients and/or may require many months of treatment. These agents may adversely affect the liver and some of these systemic medications require testing of the liver during the treatment process. In addition, patients may be allergic to oral antifungal agents.

Topical agents which would otherwise be effective against fungus have suffered from the inability to reach the fungus under the protective nail.

Nails may also be cosmetically unattractive due to fungus or other etiologies and appear discolored or abnormally thickened.

### DETAILED DESCRIPTION OF THE INVENTION

A system and method for treatment of fungus-afflicted nails, discolored nails, and abnormally thickened nails is now disclosed. The system is preferably provided as a kit having a hydrogel moist pad, a topical nanosilver treatment agent dispersed in a carrier, and most preferably a covering adapted for securing the hydrogel moist pad and topical agent in place for the treatment period.

A preferred hydrogel moist pad comprises cross-linked polyethylene oxide and water in a sheet format. A suitable hydrogel formulation is disclosed in U.S. Pat. No. 3,410,006 to King, which is herein incorporated by reference. The hydrogel moist pad provides moisture to the area to which it is applied. Upon application of the hydrogel moist pad to the skin and/or nail of a user, the hydrogel moist pad provides a moist environment. Most preferably, a hydrogel moist pad, consisting of cross-linked polyethylene oxide, and containing up to 96% water, supported upon a net of low density polyethylene is employed. The type of hydrogel useful in the invention will dry out upon prolonged exposure to air, and therefore precautions are preferably taken to avoid drying during treatment of the affected area.

The moist nature of the hydrogel moist pad provides moisture to the nail being treated during use. The nail becomes softened and pliable, and the moisture activates the fungus. This activation allows dormant fungi under the nails to be made ready for uptake of a treatment agent provided concurrently with the hydrogel moist pad to the nail. The preferred treatment agent is active in an aqueous environment and upon uptake by the active fungus, eradicates the organism.

A preferred polyethylene oxide is Union Carbide POLYOX WSR-1105. The hydrogel moist pads may be made by introducing a polymer (solid) into water, creating a feed mix. The feed mix is used to coat a web material (or scrim) and two outer linings are applied creating sheets of hydrogel. These sheets are then introduced to a high energy field which cross-links the polymers giving the hydrogel greater molecular integrity and thereby creating sheets of hydrogel. Cross linking may be accomplished through chemical cross linking known to the art. Utilizing an electron beam accelerator is preferred. Electron beam cross-linking is achieved through the introduction of the high energy field, created by the accelerated electrons, which causes the release of hydrogen atoms thereby causing carbon molecule covalent bonding. The creation of longer chains of the polymer in the gel increases its molecular integrity, giving the gel the desired characteristics. The benefits of electron beam cross-linking include: precise control of the amount of polymer cross-linking; other types allow for the continuation of cross-linking over a period of time, the ability to eliminate the need for chemical cross-linking agents which may complicate or interfere with other additives or active ingredients; and the ability to manufacture high quality hydrogels on a consistent basis.

The physical characteristics can be further modified by varying the percent of polymer cross-linking and the way in which the high energy field is delivered. There are three variables in the use of an electron beam accelerator for cross-linking of hydrogels, including time of exposure of the target material to the electron stream, the voltage (electrical potential), and amperage (strength of the electrical current).

A preferred hydrogel moist pad suitable for the system and method of the invention is commercially available from Spenco Medical Corporation (Waco, Tex.) under the trademark 2nd Skin®. 2nd Skin® hydrogel moist burn pads are provided as a hydrogel moist pad sandwiched between a first sheet of polyethylene film (clear) and a second sheet of polyethylene film (blue). For use in the system and method of this invention, one sheet of polyethylene film is removed prior to application, exposing the moist hydrogel which is applied to the affected nail as further described below.

A topical agent useful in the system of the invention employs a nanosilver treatment agent. By “nanosilver treatment agent” it is meant specifically a silver composition made according to U.S. Pat. Nos. 7,135,195, 6,743,348 or 6,214,299 which are herein incorporated by reference. Most preferably, a nanosilver treatment agent is a colloidal silver composition made pursuant to U.S. Pat. No. 7,135,195 and has the characteristics described therein. Specifically, silver particles which comprise an interior of elemental silver and an exterior of ionic silver oxide, and water, wherein the silver particles are placed in colloidal suspension in the water at a level of 5 to 40 ppm total silver. Preferably, more than 50% of the silver particles have a maximum dimension less than 0.015 micrometers.

The nanosilver treatment agent is stably suspended in an aqueous solution or water-based gel. The formulation is aqueous and crystal clear.

The form of nanosilver treatment agent is metallic silver having an outer coating of a stoichiometric combination of silver (I) and silver (III) oxide. The analysis of the silver content in the silver compositions of this invention may be done by atomic absorption (AA), inductively coupled plasma/atomic emission (ICP/AES), or other techniques known to one of ordinary skill in the art to be sensitive to silver in the appropriate concentration range.

The concentration of the nanosilver treatment agent in the carrier is from about 5 to about 40 parts per million (ppm). A preferred concentration is from about 10 to about 40 ppm. More preferably, a concentration of about 12 to 24 ppm is employed. The higher concentrations may cause irritation to some patients, so it is preferred to use a lower concentration which will still have efficacy for the desired application. A most preferred concentration having efficacy for nail treatment is 12 ppm.

The nanosilver treatment agent is preferably suspended in a pharmaceutical carrier suitable for topical application. Most preferably, the carrier is a gel. A preferred gel is an amorphous water-based gel. Moisture is managed using an aqueous base combined with a blend of hydrophilic substances. A most preferred gel comprises purified water, triethanolamine (TEA) and carbomer.

In a preferred regimen for use of the system of the invention, a user may first prepare the nail for treatment. This may include trimming the nail afflicted with fungus and scraping debris from under the nail. After cleaning the area, or after a shower or bath, the topical nanosilver treatment agent dispersed in a carrier is applied to the surface of the nail and the skin at the base of the nail as much as possible.

The nail and nanosilver treatment agent will then be covered with a hydrogel moist pad. The hydrogel moist pad can be customized for the user so that it covers the entire surface of the nail, most preferably over the top of the nail and also over the topical agent pushed under the nail. In another embodiment, pre-cut sheet portions adapted for different sizes of fingernails or toenails can be supplied.

Ideally, the hydrogel moist pad should overlap only about 2 mm on the skin surrounding the nail to avoid prolonged hydration of the sides and bottom of the finger or toe.

If covered with a protective film as supplied, the protective film will be removed from one side of the hydrogel moist pad to expose the hydrogel. The exposed moist hydrogel is placed on the nail to which the topical agent has already been applied. The other layer of the protective film is preferably left in place on the hydrogel moist pad. The protective film left in place will assist in keeping the moisture in the hydrogel moist pad and avoiding desiccation.

In order to secure the hydrogel moist pad and topical agent to the nail, in a preferred embodiment a kit is supplied that, in addition to a hydrogel moist pad and a nanosilver treatment agent, contains a bandage or tape for application to the finger or toe and nail. A bandage can be pre-cut in a shape adapted for a human digit. Some suitable shapes for such a bandage are “T-shaped” and “L-shaped.” A T-shaped bandage is disclosed in U.S. Pat. No. 2,440,235 to M. Solomon which is herein incorporated by reference. An example of an “L-shaped” bandage is disclosed in U.S. Pat. No. 3,880,159 to Diamond which is herein incorporated by reference.

It is desirable that the bandage have a tab which may be folded over the tip and nail of the digit. For example, when an L-shaped adhesive bandage, which will function as an occlusive or semi-occlusive covering, is employed, the shorter end of the L is wrapped over the tip of the nail covering the clear film of the hydrogel which overlies the nanosilver treatment agent. The edges of the adhesive are then sealed to hold in moisture. The longer end will wrap around the digit and secure the hydrogel moist pad in place. In the case of a T-shaped bandage, a first leg of the T may be folded over the tip and nail of the finger or toe and the other two legs wrapped around the digit to secure said first leg, as illustrated in U.S. Pat. No. 2,440,235, FIG. 4. In the case of the instant invention, a nanosilver treatment topical agent will first be applied to the nail, followed by a hydrogel moist pad, and then the first leg of the T folded over the hydrogel moist pad. The other two legs are then folded over the first leg to further secure it in place. Another bandage which may be used comprises two strips of adhesive that can be crossed over in application. In such case, the first strip of adhesive is placed over the film of the hydrogel dressing. It is secured around the toe and adhesively secured to the underside of toe. A second strip of adhesive is preferably placed over the length of the toe. It should be perpendicular to the first tape and is secured underneath the tip of the toe at one end and also on top of the foot to completely seal in the topical agent.

Alternatively, other pre-shaped bandages suitable to seal the topical agent covered with the hydrogel moist pad can be utilized.

The bandage is preferably made of a material which will secure the topical agent covered with the hydrogel moist pad in place on the nail and will prevent or slow desiccation of the hydrogel moist pad. A moisture impermeable flexible film or fabric is preferred. In a preferred embodiment, the bandage is made of a spun lace polyester non-woven fabric, laminated with an adhesive coating and release paper, and pre-cut into a desired shape. Upon use, the release paper is removed which exposes the adhesive coating which is then applied to the hydrogel moist pad/nanosilver treatment agent assembly on the nail of the human digit, thereby securing the assembly in place and providing a seal to prevent moisture loss.

In one regimen, the treatment is left in place for approximately 24 hours then removed. The area is then cleaned and a second application applied. This is repeated every 24 hours for three to ten consecutive days. Preferably, the treatment is conducted for at least seven days and most preferably ten days.

The treatment area is then left untreated for at least several days and preferably up to three weeks.

Treatment with the nanosilver treatment agent and hydrogel moist pad is then preferably resumed for three to ten consecutive days according to the procedure for the first treatment.

The treatment and waiting period is then repeated for one to four cycles, preferably one to six cycles, or until the toenail is healthy and free from apparent fungal affliction and/or discoloration or abnormal thickening.

Without wishing to be bound to any theory, it is believed that providing a nanosilver treatment agent gel which is dispersed in water and held in suspension acts in concert with the hydrogel moist pad dressing, which has a high water content, to disperse the nanoparticles of silver to the tissues.

It is believed that the charged silver emits a unique energy similar to that of ultraviolet light which is transferred to the entire body of water. If a solution or gel containing this silver nanoparticle hydrosol dries up, the silver is no longer active in this application. This silver hydrosol is only active when it is wet and this is the cornerstone of this synergistic effect.

It is believed that the hydrogel moist pad also acts to soften the nail and allows delivery of the antifungal to the area underneath the nail which has previously only been successfully achieved consistently with systemic therapy.

The water in the hydrogel moist pad and the moist environment provided by the hydrogel moist pad and maintained by the occlusive or semi-occlusive dressing, helps to activate the fungal spores so that they can be eliminated. Dormant spores are very difficult to eradicate until they become active.

Below is a table that compares the efficacy of three regimens for treating human nails: (1) a nanosilver treatment agent gel preparation (SILVERSOL®, American Silver, LLC);(2) a hydrogel moist pad (2ND SKIN® moist burn pad, Spenco Medical Corporation, Waco, Tex.), and (3) a combination of (1) and (2). Each regimen was used to treat cadaver toe nails inoculated with Trichophyton rubrum (ATCC 28177). A protocol was established to study the penetration of various treatments on the inoculated cadaver toe nails. Toenails were treated with daily applications of the various treatments described above. A control (no treatment) was also run simultaneously.

Organism Preparation: Sabouraud Dextrose Broth (SDB)(5 mL) was inoculated with Trichophyton rubrum in a test tube and allowed to grow until growth appeared. Ideal growth conditions were 30° C.±2° C. with humidity for 3-7 days. To quantify the growth, the SDB growth was used to inoculate a Sabouraud Dextrose Agar (SDA) plate. The organisms were incubated at 30° C.±2° C. with humidity for a minimum of 7 days or until substantial growth developed. A solution of sterile 0.1% peptone was dispensed onto the top of the petri dish with growth. Sufficient volume was dispensed to cover the petri dish with growth. Colonies were harvested by scraping the agar plate with an inoculating loop (sterile). The peptone/Trichophyton suspension was pipetted into a clean, sterile test tube and vortexed sufficiently to generate a homogenous suspension. A standard plate count for Trichophyton rubrum was performed to determine the number of colony forming units (CFU/mL). Serial tenfold dilutions using 0.1% Peptone for dilutions was done and each dilution plated in duplicate using Sabouraud Dextrose Agar (SDA). The dilution tubes were stored in a 15 mL sterile centrifuge tubes at 4° C. for later use. SDA plates were incubated at 30° C.±2° C. with humidity until colonies were visible.

After determining the concentration of Trichophyton in the dilution tubes, the corresponding dilution tube that equates to a concentration of 10⁶-10⁷ CFU/mL was used for inoculating the test toenails. Whole, great toe nails from cadavers were obtained. Nails were procured from an authorized source, with certificates of compliance or equivalent for the nails.

Toenails were screened and by visual inspection found to be free of fungus (not thick, no peeling noticeable, and no excess debris). Scales or other imperfections were removed by filing.

The nail thickness was measured and recorded using calibrated calipers or equivalent. The thickness of the thickest portion of the nail (most often the center of the nail) was recorded.

All nails were sterilized via 70% isopropyl alcohol (IPA) prior to use. The toenails were dipped in the IPA solution for a minimum of ten seconds and allowed to dry sufficiently. It was found that sufficient drying under a hood can be achieved in approximately 2 hours or longer without a hood.

Components: A nanosilver treatment agent containing either 12 ppm or 24 ppm silver (manufactured by American Silver, LLC (Alpine, Utah)) made pursuant to U.S. Pat. No. 7,135,195, a hydrogel moist pad (2nd Skin® moist burn pad, manufactured by Spenco Medical Corporation, Waco Tex.) and an adhesive knit dressing (Spenco Medical Corporation, Waco Tex.) were used for in the testing.

Organism Inoculation Procedure: Four (4) treatment groups were prepared to include five (5) toenails per group. Nails were assigned to a treatment group, or control group, and this information was recorded for the final report. Each nail was inverted so that the concave side faced up and each nail was inoculated with aliquots of 0.1 mL of 10⁶-10⁷ organisms per mL of the T. rubrum. All nails were placed in individual unfilled sterile petri dishes having a lid (12 dishes total) and placed under a hood to dry for 24 hours with the concave side facing up.

Test Article Application Procedure: Application of the three (3) treatment regimens were performed daily for 10 consecutive days on corresponding test groups of toenails. Treatment was performed every 24 hours±30 minutes.

GROUP 1: For the nanosilver treatment agent test group; 0.2-0.3 mL of the gel was applied to the top portion (convex side) of each nail to be tested. A sterile syringe or equivalent was used to apply the nanosilver treatment agent in a consistent and uniform manner. Care was taken to ensure that none of the nanosilver treatment agent seeped to the inoculated portion of the nail on the underside. The nail was replaced in the petri dish with the convex side facing up with the treatment.

For the next day's treatment, a sterile swab was dipped in sterile water, or equivalent, to remove any residual nanosilver treatment agent from the previous treatment before the same treatment was reapplied. This process was repeated daily for 10 consecutive days.

All test samples were placed in the environmental chamber set at 30° C.±2° C. with humidity for the duration of the study.

GROUP 2: For the hydrogel moist pad test group, a one inch by one inch (2.5 cm by 2.5 cm) section of a hydrogel moist pad was used to cover the surface of the top portion (convex side) of each nail to be tested. The hydrogel moist pad was removed from the sealed foil pouch in which it was packaged, and the blue liner removed from one face of the pad to expose a hydrogel surface. A clear liner adhered to the other face of the pad was left in place. The pad was trimmed if necessary to ensure it did not overlap the sides of the toe nail, and it was placed with the exposed hydrogel surface in direct contact with the nail surface. The opposite side of the hydrogel moist pad, still adhered to a clear liner, was not in contact with the nail. Each day the previous hydrogel moist pad was removed and a fresh hydrogel pad placed in the same location and in the same manner.

For the next day's treatment, a sterile swab dipped in sterile water, or equivalent, was used to remove any residual hydrogel from the previous treatment before reapplying the same treatment. This process was repeated daily for 10 consecutive days. All test samples were placed in the environmental chamber set at 30° C.±2° C. with humidity for the duration of the study.

GROUP 3: For the test group of the combination of the treatment of group 1 and the treatment of group 2, approximately 0.2 mL to 0.3 mL of the nanosilver treatment agent was applied to the top portion (convex side) of each nail to be tested. A sterile syringe or equivalent was used to apply the nanosilver treatment agent in a consistent and uniform manner, in the same manner as for the Group 1 testing. A 2.5 by 2.5 cm square (one inch by one inch) of the hydrogel moist pad as employed in Group 2 testing was then applied in the same way as applied for Group 2 testing. The hydrogel pad face from which the blue liner was removed was applied directly to the nanosilver treatment agent. The nail with the two treatment groups was put back into the petri dish and an adhesive knit bandage applied to the entire assembly. Gentle pressure was applied to the adhesive knit dressing, but not enough pressure to squeeze out the nanosilver treatment agent. The adhesive knit was sufficiently large enough to secure the perimeter of the entire nail directly to the petri dish.

For the next day's treatment, the adhesive knit and hydrogel moist pad was removed and a sterile swab was dipped in sterile water, or equivalent, to remove any residual hydrogel from the previous treatment before reapplying the same treatment.

This process was repeated daily for 10 consecutive days. All test samples were placed in the environmental chamber set at 30° C.±2° C. with humidity for the duration of the study.

GROUP Control: The three (3) remaining inoculated nails were left in their respective petri dishes as positive controls. They were kept with the convex side facing up. All test samples were replaced in the environmental chamber set at 30° C.±2° C. with humidity for the duration of the study.

Article Test Procedure: Daily observation was performed on all groups.

All four (4) test groups were tested for bio burden at Day 11 following the 10 consecutive treatments. 10 mL D/E Broth with inactivators was used to inactivate the antimicrobial from the test articles. Each toenail was placed in a 10 mL D/E Broth tube (10⁻¹ dilution) and vortexed a minimum of 30 seconds.

Serial tenfold dilutions (1 mL to 9 mL 0.1% Peptone) from 10⁻² through 10⁻⁵ were performed using 0.1% Peptone. Each dilution was mixed thoroughly by vortexing.

Each dilution was plated in duplicate using SDA-Plus inactivators. SDA plates were incubated at 30° C.±2° C. and 65-75% until colonies were visible. The colonies were enumerated and results tabulated in Table 1 for the 24 ppm nanosilver treatment agent gel.

TABLE 1
Organism Trichophyton rubrum ATCC 28177
Initial Organism Count 1.74 × 10⁶

| | | Final Count | Log | |
|---|---|---|---|---|
| Group | Sample ID | CFU/ml | reduction | % reduction |
| Positive Control Group | Toe nail-1 | 1.23 × 10⁷ | −0.84936 | −606.896552% |
| | Toe nail-2 | 2.04 × 10⁷ | −1.06908 | −1072.4137% |
| | Toe nail-3 | 1.23 × 10⁷ | −0.84936 | −606.896552% |
| | Average | 1.50 × 10⁷ | −0.93554 | −762.068966% |
| | Standard Deviation | 3.82 × 10⁶ | | |
| Group I - 24 ppm | Toe nail-1 | 1.05 × 10⁵ | 1.21936 | 93.965517% |
| Nanosilver treatment agent gel | Toe nail-2 | 8.25 × 10⁴ | 1.32410 | 95.258621% |
| | Toe nail-3 | 4.45 × 10⁵ | 0.59219 | 74.425287% |
| | Average | 2.11 × 10⁵ | 0.91661 | 87.883142% |
| | Standard Deviation | 2.03 × 10⁵ | | |
| Group II - Hydrogel moist pad | Toe nail-1 | 1.26 × 10⁵ | 1.14018 | 92.758621% |
| | Toe nail-2 | 6.55 × 10³ | 2.42431 | 99.623563% |
| | Toe nail-3 | 9.15 × 10⁴ | 1.27913 | 94.741379% |
| | Average | 7.47 × 10⁴ | 1.36733 | 95.707854% |
| | Standard Deviation | 6.15 × 10⁴ | | |
| Group III - 24 ppm Nanosilver | Toe nail-1 | Less than 10 | 5.24055 | 99.999425% |
| treatment agent in combination | Toe nail-2 | Less than 10 | 5.24055 | 99.999425% |
| with hydrogel moist pad | Toe nail-3 | 2.1 × 10² | 3.91833 | 99.987931% |
| | Average | 7.67 × 10¹ | 4.35594 | 99.995594% |
| | Standard Deviation | 1.15 × 10² | | |

The results shown in Table 1 demonstrate a synergistic effect when nanosilver treatment agent is used in combination with a hydrogel moist pad to treat toenails infected with T. rubrum. As would be expected, the positive control group exhibited growth of the organism. With use of the 24 ppm nanosilver treatment agent alone, an average 0.92 log reduction was observed. When the hydrogel moist pad alone was used to treat the experimental cadaver toenails, a 1.37 log reduction was observed. However, when the treatments were used in combination, a 4.4 log reduction was observed which is much greater than an additive effect.

The data in Table 2 was generated by using the protocol described above for Table 1 with some exceptions. Namely, a combination of 12 ppm nanosilver treatment agent and hydrogel moist pad as well as a combination of 24 ppm nanosilver treatment agent in combination with a hydrogel moist pad was tested. However, instead of treating the subject toenails for ten consecutive days, the toenails were treated for eight consecutive days, no treatment was provided on days 9 and 10, and then treatment was resumed with daily cleanings and fresh application of nanosilver treatment agent and hydrogel moist pads for two additional days. The bioburden was then measured. The results in Table 2 show efficacy of 12 ppm and 24 ppm nanosilver treatment agent in combination with the hydrogel moist pad, although the log reduction was not as pronounced as the results in Table 1, perhaps due to the interruption in treatment at days nine and ten.

TABLE 2
Organism Trichophyton rubrum ATCC 28177
Initial Organism Count 1.03 × 10⁶

| | | Final Count | Log | |
|---|---|---|---|---|
| Group | Sample ID | CFU/nail | reduction | % reduction |
| Group 1 - 12 ppm | Toe nail-1 | 2.1 × 10⁵ | 0.69062 | 79.611650% |
| Nanosilver treatment agent | Toe nail-2 | 6.8 × 10⁵ | 0.18033 | 33.980583% |
| | Toe nail-3 | 1.2 × 10⁵ | 0.93366 | 88.349515% |
| | Average | 3.4 × 10⁵ | 0.48564 | 67.313916% |
| | Standard Deviation | 3.01 × 10⁵ | | |
| Group 2 - Hydrogel Moist Pad | Toe nail-1 | 1.9 × 10⁶ | −0.26592 | −84.466019% |
| | Toe nail-2 | 1.6 × 10⁶ | −0.19128 | −55.339806% |
| | Toe nail-3 | 1.1 × 10⁶ | −0.02856 | −6.796117% |
| | Average | 1.5 × 10⁶ | −0.17280 | −48.867314% |
| | Standard Deviation | 4.04 × 10⁵ | | |
| Group 3 - 12 ppm Nanosilver | Toe nail-1 | 3.3 × 10⁴ | 1.49432 | 96.796117% |
| treatment agent and hydrogel | Toe nail-2 | 3.7 × 10⁴ | 1.44464 | 96.407767% |
| moist pad combination | Toe nail-3 | 3.3 × 10⁴ | 1.49432 | 96.796117% |
| | Average | 3.4 × 10⁴ | 1.47712 | 96.666667% |
| | Standard Deviation | 2.31 × 10³ | | |
| Group 4 - 24 ppm Nanosilver | Toe nail-1 C101213 | 7.5 × 10² | 2.97248 | 99.893459% |
| treatment agent and hydrogel | Toe nail-2 C101226 | 5.0 × 10² | 3.14858 | 99.928973% |
| moist pad combination | Toe nail-3 C101371 | 9.5 × 10² | 2.86982 | 99.865049% |
| | Average | 7.3 × 10² | 2.98224 | 99.895827% |
| | Standard Deviation | 2.25 × 10² | | |
| Group 5 - Positive Control: | Toe nail-1 | 1.0 × 10⁷ | −0.98716 | −870.873786% |
| No Treatment | Toe nail-2 | 9.4 × 10⁶ | −0.96029 | −812.621359% |
| | Toe nail-3 | 8.3 × 10⁶ | −0.90624 | −705.825243% |
| | Average | 9.23 × 10⁶ | −0.95252 | −796.440129% |
| | Standard Deviation | 7.04 × 10⁵ | | |

In order to test the concentration of nanosilver treatment agent that is effective in killing various fungal organisms, kill studies were conducted. Table 3 shows the effect of 12 ppm nanosilver treatment agent according to the invention.

TABLE 3

| | | Positive Control | | | |
|---|---|---|---|---|---|
| | Negative | (T. rubrum) | Candida | Trichophyton | Trichophyton |
| | Control | 10% Bleach | albicans | rubrum | mentagrophytes |
| Organism | Sterile Water | Solution | ATCC 10231 | ATCC 28188 | ATCC 9533 |
| Organism Count | 0 | 1.9 × 10⁶ | 1.3 × 10⁶ | 1.9 × 10⁶ | 1.5 × 10⁶ |
| Neutralization | N/A | 3.3 × 10⁵ | 1.3 × 10⁶ | 1.8 × 10⁶ | 1.4 × 10⁶ |
| Initial Count | 0 | 2.0 × 10⁵ | 1.3 × 10⁶ | 1.8 × 10⁶ | 1.4 × 10⁶ |
| 15 Minute Counts | 0 | 2.0 × 10⁴ | 2.2 × 10³ | 1.0 × 10⁴ | 2.8 × 10³ |
| 1 Hour Counts | 0 | 1.3 × 10³ | 2.3 × 10² | 4.9 × 10³ | 6.2 × 10² |
| 24 Hour Counts | 0 | 0 | 2.0 × 10² | 1.8 × 10³ | 2.8 × 10² |
| 48 Hour Counts | 0 | 0 | 0 | 7.2 × 10² | 9.0 × 10¹ |
| 7 Day Counts | 0.5 × 10¹ | 0 | 0 | 7.5 × 10¹ | 0 |
| Percent | N/A | 10.53 | 100 | 94.74 | 93.33 |
| Neutralization | | | | | |

Log Reduction

| | | | | | |
|---|---|---|---|---|---|
| 15 Minute Counts | N/A | 1.00000 | 2.77152 | 2.25527 | 2.69897 |
| 1 Hour Counts | N/A | 2.18709 | 3.75222 | 2.56508 | 3.35374 |
| 24 Hour Counts | N/A | 5.30103 | 3.81291 | 3.0000 | 3.69897 |
| 48 Hour Counts | N/A | 5.30103 | 6.11394 | 3.39794 | 5.19189 |
| 7 Day Counts | N/A | 5.30103 | 6.11394 | 4.38021 | 6.14613 |

Percent Reduction

| | | | | | |
|---|---|---|---|---|---|
| 15 Minute Counts | N/A | 90.000000% | 99.830769% | 99.444444% | 99.800000% |
| 1 Hour Counts | N/A | 99.350000% | 99.982308% | 99.727778% | 99.955714% |
| 24 Hour Counts | N/A | 100.00% | 100.00% | 99.900000% | 99.980000% |
| 48 Hour Counts | N/A | 100.00% | 100.00% | 99.960000% | 99.999357% |
| 7 Day Counts | N/A | 100.00% | 100.00% | 99.995833% | 100.00% |

A kill study was performed with 24 ppm nanosilver treatment agent and the results are shown in Tables 4 and 5 below. From the results of the kill studies, it was concluded that a 12 ppm concentration of nanosilver treatment agent is an effective kill agent. It was also concluded that 24 ppm is a very strong kill agent, and that concentrations in excess of 24 ppm would not be necessary in the application.

TABLE 4

| | Trichophyton | |
|---|---|---|
| | mentagrophytes | Trichophyton rubrum |
| | ATCC 9533 (D/E Broth) | ATCC 28188 (D/E Broth) |
| Organism | CFU/1 mL | CFU/1 mL |
| Organism Count | 6.4 × 10⁷ | 7.9 × 10⁷ |
| Initial Count | 4.4 × 10⁴ | 4.0 × 10⁴ |
| 24 Hour Count | 5.0 × 10² | All 0 |
| 48 Hour Count | All 0 | All 0 |
| 72 Hour Count | All 0 | All 0 |
| 7 Day Count | All 0 | All 0 |

TABLE 5

| | Trichophyton mentagrophytes | Trichophyton rubrum |
|---|---|---|
| | ATCC 9533 | ATCC 28188 |
| Organism | CFU/1 mL | CFU/1 mL |
| Organism Count | 4.9 × 10⁶ | 9.6 × 10⁶ |
| Initial Count | 4.8 × 10⁶ | 9.3 × 10⁶ |
| 5 Minute Count | 6.0 × 10² | 1.3 × 10² |
| 15 Minute Count | 0 | 4.5 × 10¹ |
| 1 Hour Count | 0 | 3.0 × 10¹ |
| 24 Hour Count | 0 | 0 |
| 48 Hour Count | 0 | 0 |
| 7 Day Count | 0 | 0 |
| 30 Day Count | 0 | 0 |

In an alternative embodiment, a hydrogel moist pad is impregnated with nanosilver treatment agent particles, said particles as described and claimed in U.S. Pat. Nos. 7,135,195, 6,743,348 and 6,214,299, and the impregnated sheet is applied to a nail to treat fungus, discoloration, or unnatural nail thickening. An adhesive bandage is preferably employed to keep the impregnated hydrogel moist pad in place on the nail during treatment.

In still another embodiment, a treatment system is supplied which comprises a hydrogel moist pad having a border edge, an adhesive secured to said hydrogel moist pad and extending outwardly from said border edge. The portion of said adhesive which extends outwardly is secured to release paper as supplied. In use, the release paper is removed and the adhesive is exposed and applied over the hydrogel moist pad to secure the hydrogel moist pad to the affected area. In this embodiment, the hydrogel moist pad defines an indentation therein, said indentation pre-loaded with a nanosilver treatment agent. Alternatively, the hydrogel moist pad is impregnated with nanosilver treatment agent. The treatment system can be applied to the area to be treated in one step.

The nanosilver treatment agent can also be impregnated into a substrate. The impregnated substrate may be placed in contact with the afflicted site, covered with a hydrogel moist pad and an occlusive dressing as described above. The impregnated substrate will release the nanosilver treatment agent upon hydration by the hydrogel moist pad. A substrate may be a non-woven or woven material, or other material compatible with the application.

### EXAMPLE 1

A 43 year old healthy male presented with a discolored thickened right great toenail consistent with severe onychomycosis. The subject was instructed to trim the nail, clean out debris from under the nail and shower before applying nanosilver treatment agent to the top of the nail and under the tip of the nail. The gel contained 24 ppm of silver and the amount applied was 0.4-0.5 mL. A hydrogel moist pad square (2nd Skin® hydrogel moist burn pad, available from Spenco Medical Corporation, Waco, Tex.) was applied over the nanosilver treatment agent and secured to the toe with an occlusive adhesive. This process was undertaken daily for 7 days followed by a three week rest. Within 3 weeks, a physician observed that the nail was markedly improved. Within 60 days, healthy unaffected new nail was observed growing in at the base of the nail.

### EXAMPLE 2

A 47 year old healthy female presented with discoloration and thickening of the right and left great toenails consistent with moderate onychomycosis. The percentage of nail that was abnormal was 50% on the left and 20% on the right. The subject was instructed to trim the nail, clean out debris from under the nail and shower before applying the nanosilver treatment agent to the top of the nail and under the tip of the nail. The gel contained 12 ppm of silver and the amount applied was 0.4-0.5 mL. A hydrogel moist pad square from Spenco Medical Corporation was applied over the nanosilver treatment agent and secured to the toe with an occlusive adhesive. The process was undertaken daily for 10 days followed by a three week rest. This was repeated for 3 consecutive months. The physician observed that both nails were markedly improved and appeared to be 100% clear on clinical exam 120 days after the applications were started.

### EXAMPLE 3

A ten day treatment regimen using 12 ppm nanosilver treatment agent in combination with a hydrogel moist pad is used in order to hydrate and treat thick nails or those with 25 to 100 percent of nail discoloration.

The ten day regimen is repeated each month for three to six consecutive months.

### EXAMPLE 4

A group of patient afflicted with toenail fungus was treated with 24 ppm nanosilver treatment agent in combination with a hydrogel moist pad and adhesive bandage securing the same for 7 days over 4 consecutive months. The success rate of treatment was 46%.

### EXAMPLE 5

Two groups of patients afflicted with toenail fungus were treated with either 12 ppm or 24 ppm nanosilver treatment agent in combination with a hydrogel moist pad and adhesive bandage securing the same for 7 days over four consecutive months. The success rate of treatment for 12 ppm group was 50% (8 out of 16 nails) while the success rate for the 24 ppm group was 75% (3 out of 4 nails)

### EXAMPLE 6

Two groups of patients were treated with either 12 ppm or 24 ppm nanosilver treatment agent in combination with a hydrogel moist pad and adhesive bandage securing the same for 10 days of three consecutive months. The success rate for the 12 ppm group was 63.6% (7 out of 11 nails) and the success rate for the 24 ppm group was 40% (2 out of 5 nails).

1-32. (canceled)

33. A method for treatment of a nail, said method comprising:
(a) providing a hydrosol gel having a topical nanosilver treatment agent suspended in an amorphous waterbased gel carrier;
(b) applying said hydrosol gel to said nail;
(c) covering said hydrosol gel with a hydrogel moist pad;
(d) securing said hydrogel moist pad to said nail with a substantially semi-occlusive covering to substantially seal the treatment agent and hydrogel moist pad to the nail;
(e) maintaining the application of said treatment agent as applied to said nail for a minimum appropriate time interval to remedy said nail having fungus, discoloration or unusual thickness conditions; and,
(f) repeating application of the treatment steps (a) to (e) one or more times to further remedy said nail having fungus, discoloration or unusual thickness conditions.

34. The method of claim 33, wherein said topical nanosilver treatment agent in said amorphous water-based gel carrier has a concentration of said nanosilver treatment agent from about 5 ppm to about 40 ppm.

35. The method of claim 33, wherein said topical nanosilver treatment agent in said amorphous water-based gel carrier has a concentration of said nanosilver treatment agent from about 12 ppm to about 24 ppm.

36. The method of claim 33, wherein said amorphous water-based gel carrier comprises purified water, triethanolamine (TEA) and carbomer.

37. The method of claim 33, wherein said hydrogel in said hydrogel moist pad has cross-linked polyethylene oxide and water.

38. The method of claim 33, wherein said hydrogel moist pad is secured with a substantially occlusive covering to substantially seal the treatment agent and hydrogel moist pad to the nail and to prevent moisture loss.

39. The method of claim 33, wherein said nail is treated according to steps (a) through (f) for a first time period of at least three days, no treatment is applied to said nail for a second time period of at least one week, and said nail is treated according to steps (a) through (f) for a third time period of at least three days.

40. The method of claim 33, wherein said treatment according to steps (a) through (f) is repeated four to six times.

41. The method of claim 33, wherein said time interval is 24 to 72 hours.

42. The method of claim 39, wherein the treatments conducted during said first, second, and third time periods are repeated one to six times to treat said nail.

43. The method of claim 42, wherein the treatments conducted during said first, second, and third time periods take at least one month to complete.

44. A method for treatment of a nail, comprising:
(a) providing a substrate that is impregnated with a nanosilver treatment agent;
(b) applying said substrate impregnated with a nanosilver treatment agent to said nail;
(c) covering said substrate with a hydrogel moist pad;
(d) securing said substrate and said hydrogel moist pad to said nail with a substantially semi-occlusive covering to substantially seal the substrate and hydrogel moist pad to the nail;
(e) keeping said treatment agent applied to said nail for a minimum appropriate time interval to remedy said nail having fungus, discoloration or unusual thickness conditions; and,
(f) repeating application of the treatment steps (a) to (e) one or more times to further remedy said nail having fungus, discoloration or unusual thickness conditions.

45. The method according to claim 44, wherein said substrate is a woven fabric that will release said nanosilver treatment agent upon hydration with said hydrogel moist pad.

46. The method according to claim 44, wherein said substrate is a non-woven fabric that will release said nanosilver treatment agent upon hydration with said hydrogel moist pad.

47. The method of claim 44, wherein said topical nanosilver treatment agent in said substrate has a concentration of said nanosilver treatment agent from about 5 ppm to about 40 ppm.

48. The method of claim 44, wherein said topical nanosilver treatment agent in said substrate has a concentration of said nanosilver treatment agent from about 12 ppm to about 24 ppm.

49. The method of claim 44, wherein said hydrogel in said hydrogel moist pad has cross-linked polyethylene oxide and water.

50. The method of claim 44, wherein said hydrogel moist pad is secured with a substantially occlusive covering to substantially seal the treatment agent and hydrogel moist pad to the nail.

51. The method of claim 44, wherein said nail is treated according to steps (a) through (f) for a first time period of at least three days, no treatment is applied to said nail for a second time period of at least one week, and said nail is treated according to steps (a) through (f) for a third time period of at least three days.

52. The method of claim 44, wherein said treatment according to steps (a) through (f) is repeated four to six times.

53. The method of claim 44, wherein said time interval is 24 to 72 hours.

54. The method of claim 51, wherein the treatments conducted during said first, second, and third time periods are repeated one to six times to treat said nail.

55. The method of claim 54, wherein the treatments conducted during said first, second, and third time periods take at least one month to complete.

56. A method for treatment of a nail, comprising
(a) providing a hydrogel moist pad that is impregnated with a nanosilver treatment agent;
(b) applying said hydrogel moist pad impregnated with said nanosilver treatment agent to said nail;
(c) securing said hydrogel moist pad impregnated with said nanosilver treatment agent to said nail by a substantially semi-occlusive covering so as to substantially seal hydrogel moist pad to the nail;
(d) keeping said hydrogel moist pad impregnated with said nanosilver treatment agent applied to said nail for a minimum appropriate time interval to remedy said nail having fungus, discoloration or unusual thickness conditions; and,
(e) repeating application of the treatment steps (a) to (d) one or more times to further remedy said nail having fungus, discoloration or unusual thickness conditions.

57. The method of claim 56, wherein said topical nanosilver treatment agent in said hydrogel moist pad has a concentration of said nanosilver treatment agent from about 5 ppm to about 40 ppm.

58. The method of claim 56, wherein the concentration of said topical agent in said hydrogel moist pad is from about 12 ppm to about 24 ppm.

59. The method of claim 56, wherein said hydrogel in said hydrogel moist pad has cross-linked polyethylene oxide and water.

60. The method of claim 56, wherein said hydrogel moist pad is secured with a substantially occlusive covering to substantially seal the treatment agent and hydrogel moist pad to the nail.

61. The method of claim 56, wherein said nail is treated according to steps (a) through (e) for a first time period of at least three days, no treatment is applied to said nail for a second time period of at least one week, and said nail is treated according to steps (a) through (e) for a third time period of at least three days.

62. The method of claim 56, wherein said treatment according to steps (a) through (e) is repeated four to six times.

63. The method of claim 56, wherein said time interval is 24 to 72 hours.

64. The method of claim 61, wherein the treatments conducted during said first, second, and third time periods are repeated one to six times to treat said nail.

65. The method of claim 64, wherein the treatments conducted during said first, second, and third time periods take at least one month to complete.
